(19) United States
(12) Patent Application Publication
(10) Pub. No.: US 2005/0075514 A1
(43) Pub. Date: Apr. 7, 2005

(54) Process for the oxidation of organic substrates and a catalyst composition for carrying out the process

(76) Inventors: Thomas Riermeier, Nidderau-Ostheim (DE); Manfred Hoff, Kelkheim (DE); Werner Zeiss, Eurasburg (DE); Uwe Wahren, Munchen (DE); Karlheinz Drauz, Freigericht (DE)

Correspondence Address: SMITH, GAMBRELL & RUSSELL, LLP;SUITE 3100, PROMENADE II, 1230 PEACHTREE STREET, N.E., ATLANTA, GA, 30309-3592, US

(21) Appl. No.: 10/938,039

(22) Filed: Sep. 10, 2004

(30) Foreign Application Priority Data
Sep. 12, 2003 (DE) 103 42 150.5

(51) Int. Cl. C07C 239/10; C07F 15/00; C07C 29/03

(52) U.S. Cl. 564/300; 568/860; 556/136

(57) ABSTRACT

Many inorganic substrates can be oxidized with a peroxo compound, such as hydrogen peroxide. In processes which are already known, pure alkylrhenium oxides of the formula RₐRe_{b}O_{c}L_{d} are employed. Catalyst compositions such as are obtainable from the reaction of an oxorhenium compound of the formula ReOₘAₙ with an organylating agent, in particular a Grignard compound, without the preparation process comprising an expensive sublimation, crystallization or distillation, can be employed with the same activity in the oxidation reactions. In addition to oxidation processes, catalyst compositions are disclosed.

### INTRODUCTION AND BACKGROUND

The present invention relates to a process for the oxidation of organic substrates, wherein these are reacted with a peroxo compound, in particular hydrogen peroxide or a source for hydrogen peroxide, in a solvent system in the presence of an organylrhenium oxide compound as a catalyst. According to the invention, there is employed a catalyst composition such as is obtainable by the organylation of a rhenium compound of the general formula ReOₘAₙ, wherein A is an anionic leaving group, with an organometallic compound, without isolation and purification of the organylrhenium oxide compound contained in the reaction mixture. The invention also provides a catalyst composition for carrying out the process which is obtained using a Grignard compound.

Organylrhenium(VII) oxides, in particular methylrhenium trioxide, which is usually called MTO for short, have recently found increasing interest as catalysts for oxidation reactions, such as the oxidation of olefins to epoxides and diols (U.S. Pat. No. 5,166,372), oxidation of aromatics to quinones (DE 44 197 99), oxidation of aromatic amines to nitroso compounds or N-oxides and isomerizations and metathesis of olefins (WO 91/14665). An overview of the preparation, structures and uses of rhenium(VII) oxo and imido complexes is given by C. C. Romão and W. A. Herrmann et al. in Chem. Rev. 1997,97, 3197-3246.

Three synthesis routes have become established for the preparation of alkylrhenium trioxide, such as MTO as the parent substance of the organylrhenium trioxides: A first route comprises direct alkylation of dirhenium heptoxide (Re₂O₇) with tetraalkyltin or dialkylzinc (W. A. Herrmann, J. Organomet, Chem. 1995, 500, 149-174). In this reaction, however, 50% of the rhenium is converted into non-reactive trialkylstannyl perrhenate or zinc perrhenate. A considerable disadvantage of this process is that the alkyltin compounds, which are in themselves very active, are highly toxic.

A second route comprises alkylation of Re₂O₇ in the presence of trifluoroacetic anhydride, as a result of which the formation of rhenium-containing by-products, that is to say perrhenates, is indeed largely avoided, but on the other hand the water-sensitive and expensive anhydride makes the process more expensive. Here also, again in general toxic tin-alkyls are employed for the alkylation.

Finally, according to Herrmann et al. (Angew. Chem. 1997,109,2767-2768) MTO can be produced in a third process without exclusion of moisture by reaction of perrhenates of the formula M(ReO₄) or M′(ReO₄)₂, wherein M is an alkali metal, NH₄, Ag or (CH₃)₃Sn and M′ is Zn or Ca, with tetramethyltin and trimethylchlorosilane. A disadvantage again is the requirement of having to employ a volatile highly toxic alkylating agent. Furthermore, the MTO-containing reaction mixture must be filtered and then freed from the solvent under reduced pressure, and finally the crude MTO must be purified by sublimation under a high vacuum, as a result of which the preparation becomes more expensive.

According to WO 98/47907, catalytically active compounds of the formula RₐRe_{b}O_{c}L_{d}, wherein R is an organyl radical, a is an integer from 1 to 6, b is an integer from 1 to 4, c is an integer from 1 to 12, d is an integer from 0 to 4 and L is a Lewis-base ligand and Re is penta- to heptavalent, can generally be produced by the abovementioned process principle. Organylating reagents which are mentioned in this document are organometallic compounds of Sn, Zn, Al, Mg, Li, Cu, Cd and Hg, but preferably those of Sn and Zn. Although organometallic compounds of magnesium are mentioned, there is no indication of employing a Grignard compound as the sole organylating agent. In fact, the teaching of this document is only that the zinc alkylating agents which are preferably to be employed (ZnR₂) can be formed by an in situ reaction from ZnCl₂ and alkyllithium (RLi) or a Grignard compound (RMgX). However, the need to have to employ both a zinc salt and in addition a Grignard compound makes the process more expensive. Although the perrhenates obtained during the working up of reaction mixtures for the purpose of recovery of Re catalysts do not have to be purified before the organylation, the organylrhenium oxide to be employed as a catalyst must itself be purified by sublimation or by crystallization.

In the ethenolytic metathesis of olefins according to WO 91/14665 also, a rhenium catalyst of the abovementioned genus RₐRe_{b}O_{c}, such as (CH₃)ReO₃ or (CH₃)₄Re₂O₄, although bonded to an oxidic support here, is always employed in the purified form. Indications of using as the catalyst composition a composition obtained in the preparation of the organylating agent, without an expensive purification operation, are not to be found in this document.

In respect of the use of rhenium catalysts of the abovementioned general formula RₐRe_{b}O_{c}L_{d} as an oxidation catalyst, reference is also made to the EP patent 0380 085. The content of this document is relied on and included in its entirety herein. The preparation process described in this document for the catalyst to be employed, MTO or an aminic complex of MTO, again comprises a vacuum sublimation of the MTO. As an alternative to MTO, solid tetramethyltetraoxodirhenium ((CH₃)₄Re₂O₄) is employed as the starting compound.

In example 42 of EP 0 380 085 B1 (trimethylsilyl)methylmagnesium chloride, that is to say a Grignard compound, is used: This compound is reacted with the bipyridine complex of methyl(oxo)dichlororhenium(V); the reaction mixture which has been freed from solvents is separated by chromatography and the trimethyloxorhenium complex is crystallized out of the appropriate fraction. Indications of producing a reaction mixture by a reaction of an oxorhenium compound of the formula ReOₘAₙ with a Grignard reagent and of using this without an expensive purification operation, excluding filtration or extraction and distilling off of solvents, as a catalyst in oxidation reactions, in the metathesis of olefins and in other reactions catalysed with organylrhenium oxides are not to be found in this document.

In the review article cited in the introduction (Chem. Rev. 1997), it is stated on page 3210 that anionic substitution of compounds of the type XReO₃, such as ClReO₃, Re₂O₇ or Me₃SiOReO₃, with lithium-, magnesium- and aluminium-alkyls is advised against, because reduction reactions occur with these to a greater extent than with zinc-alkyls. Finally, the teaching on page 3211 of this document is that isopropylzinc chloride, which is structurally the same as a Grignard reagent, is unreactive towards dirhenium heptoxide. No suggestion is given for the sole use of methylmagnesium chloride or methylzinc chloride or alpha-unbranched alkylmagnesium halides or corresponding alkylzinc halides as the organylating agent and the use of a reaction mixture comprising alkylrhenium oxide as a catalyst in oxidation reactions.

According to W. A. Herrrann and F. E. Kuhn (Acc. Chem. Res. 1997, 30, 169-180) organorhenium(VII) oxides can also be produced by catalytic oxidation of organorhenium-alkyls with H₂O₂ and iodine as a catalyst. It is also the doctrine in this document that using Grignard reagents or alkyllithium, alkylrhenium(VI) oxides of the formula R₄ReO are obtainable from OReCl₃(PPh₃)₂ or OReCl₄, in each case with subsequent oxidation of the Re(V) intermediate stage. No suggestion of alkylating a compound of the type ReOₘAₙ, wherein Re is heptavalent and A is an anionic leaving group, by means of a Grignard compound can be found in this document.

### SUMMARY OF THE INVENTION

The object of the present invention accordingly is to provide a further process for carrying out oxidation reactions of organic substrates in the presence of organylrhenium oxides and derivatives thereof, wherein a rhenium catalyst which is obtainable in a simple manner should be used, in order to reduce the total outlay for provision of the catalyst and the oxidation reaction.

A further object of the invention is to provide a catalyst composition comprising an organylrhenium oxide which can be employed in oxidation reactions without expensive purification operations.

It has been found that for the oxidation reactions in question using peroxygen compounds, such as, in particular, hydrogen peroxide or a source for this, a catalyst composition such as is obtainable in the context of the preparation of the organylrhenium oxides, without purification operations worth mentioning, in particular no sublimation, distillation or crystallization of the organylrhenium oxide, surprisingly is substantially just as active as a catalyst as an equivalent amount of the catalyst employed to date, which has been purified in an expensive manner. It was evidently assumed hitherto that the expensive purification operations on the organylrhenium oxides, such as MTO, cannot be dispensed with without having to pay the price of a corresponding deterioration in the catalytic activity of the catalyst.

The invention provides a process for the oxidation of organic substrates comprising reaction of the organic substrate, in a solvent system, with a peroxo compound, in particular hydrogen peroxide, in the presence of an organylrhenium oxide of the formula RₐRe_{b}O_{c}L_{d}, wherein R is an organyl radical, a is an integer from 1 to 6, b is an integer from 1 to 4, c is an integer from 1 to 12, d is an integer from 0 to 4, L is a Lewis-base ligand and Re is penta- to heptavalent, as a catalyst, characterized in that that a catalyst composition K1 or a catalyst composition K2, K3 or K4 obtained therefrom is employed. The preparation of K1 comprises the reaction of an oxorhenium compound of the general formula ReOₘAₙ, wherein Re is penta- to heptavalent, A represents an anionic leaving group, m represents the number 1, 2 or 3 and n represents the number 0, 1, 2 or 3, with 0.5 to 10 organyl equivalents per mole of ReOₘAₙ of an organylating agent of an element from the series consisting of Li, Na, K, Mg, Al and Zn in an aprotic solvent in the presence or absence of a Lewis-basic ligand. The preparation of K2 comprises the addition of a protic agent to a composition K1 comprising excess organylating agent, and the preparation of K3 and K4 comprises at least partial removal of the solvents and/or inorganic constituents from K1 and K2 respectively, but the preparation of K1 to K4 excludes a sublimation, crystallization or distillation of the organylrhenium oxides of the formula RₐRe_{b}O_{c}L_{d} contained therein.

Preferred embodiments of the process in respect of the preparation of the catalyst composition to be used and the oxidation of specific substrates are described below.

An essential feature of the process according to the invention is that a catalyst composition K1 or a catalyst composition K2, K3 or K4 obtainable therefrom in a simple manner is employed for carrying out the oxidation. These compositions are reaction mixtures which result directly from the reaction of an oxorhenium compound with an organylating agent (K1), wherein an excess of organylating agent contained in the reaction mixture can have been destroyed by means of a protic system (K2) and solvents and/or inorganic constituents can have been separated off in a simple manner known per se (K3 and K4). The separating off of inorganic constituents can comprise, for example, a filtration or extraction, an extraction being advantageous in particular if an aqueous medium has been added to the reaction mixture after the reaction in order to destroy excess organylating agent.

An essential advantage of the invention is thus that the isolation and purification of the organylrhenium oxide active as the catalyst by expensive processes, such as sublimation, crystallization or distillation under a high vacuum, hitherto considered necessary can be dispensed with. Surprisingly, a catalyst composition K1, K2, K3 or K4 can be employed as the catalyst for oxidation reactions without having to pay the price of a reduction in the catalytic activity compared with purified organylrhenium oxide with the same amount of Re employed. It is presumed that the organylating agent has a more or less pronounced reducing action, and the catalyst composition comprises more than a single organylrhenium oxide. Organylrhenium oxides which contain rhenium in an oxidation level of less than 7 are evidently converted into catalytically active compounds of oxidation level +7 by the oxidizing agent employed in the oxidation or/and organylrhenium oxides of Re oxidation level of less than 7 contained in the catalyst composition are themselves sufficiently catalytically active.

An oxorhenium compound of the general formula ReOₘAₙ with the meaning already mentioned for the valency of Re, A, m and n is employed for the preparation of an active catalyst composition K1 or a catalyst composition K2, K3 or K4 obtained therefrom. For example, the rhenium oxides Re₂O₅, ReO₃ and Re₂O₇ are employed, the latter being particularly preferred. In the case of the oxides Re₂O₇ and Re₂O₅, the leaving anionic grouping is the perrhenate ion or rhenate ion respectively. In the case of rhenium oxide ReO₃, n is 0.

Further classes of suitable rhenium compounds for the preparation of the catalyst compositions are substances from the series consisting of HalReO₃, Hal₂ReO₂, HalReO₂, Hal₃ReO and Hal₃ReO₂, wherein Hal represents a halogen from the series consisting of fluorine, chlorine, bromine or iodine where chlorine, however, is particularly preferred.

Further classes of very particularly suitable oxorhenium compounds are, in particular, those of the general formula ReO₃—O-acyl and ReO₂—O-acyl, the class of 7-valent rhenium compounds being preferred. Acyl is preferably a grouping from the series consisting of R—C(O)—, aryl-SO₂—, —P(O)(OR′)₂, —Si(OR′)₃, —P(O)R″₂ and —SiR″₃, wherein R represents optionally fluorinated alkyl and R′ and R″ represent alkyl, cycloalkyl, arylalkyl or aryl, which can also be fluorinated. Examples of these acyl groups are acetyl, trifluoroacetyl, benzoyl and benzenesulfonyl, wherein the phenyl radical can also be substituted, such as fluorinated or methylated, trimethoxysilyl, triethoxysilyl and trimethylsilyl.

The oxorhenium compound is reacted with an organylating agent, this reaction being carried out in an aprotic solvent or solvent mixture, in particular an ether, at a temperature in the range from about −70° C. to about +80° C., preferably in the range from −25 to +60° C. and particular about 0° C. to 40° C.

Possible organylating agents for the preparation of the catalyst compositions to be used according to the invention are organylmetal compounds of alkali metals, magnesium, calcium, aluminium and zinc. According to a preferred embodiment, an organylmagnesium halide is used as the organylating agent. Examples of suitable organylating agents are LiR, NaR, KR, MgR₂, ZnR₂, AlR₃ and Al(O)R, but in particular RMgHal, wherein Hal represents a halogen, in particular chlorine. The organyl radical R denotes linear, branched or cyclic alkyl, in particular unbranched C₁- to C₄-alkyl, particularly preferably methyl; however, R can also denote benzyl or aryl, wherein the phenyl radical or aryl radical can be substituted.

According to particularly preferred embodiments, a lithium-alkyl compound or a Grignard reagent, in particular an alkylmagnesium halide, particularly preferably methylmagnesium chloride, is employed as the organylating agent. The choice of the organyl radical is made by the expert such that the catalyst composition obtained has the highest catalytic activity. Organylrhenium oxides or complexes thereof which contain one or more methyl groups as the organyl radical have a particularly high activity.

As has been stated above, oxorhenium compounds of the general formula ReOₘAₙ with heptavalent Re were hitherto not alkylated with the sole use of alkylmagnesium halides, that is to say without the roundabout route via the preparation of zinc-alkyls. However, the use of alkylmagnesium halides is particularly advantageous because this substance class can be produced and used under conditions which are easy to handle.

For organylation of the oxorhenium compound, in general 1 to 10 organyl equivalents, preferably 1 to 5 equivalents and in particular >1 to 3 organyl equivalents are employed per mole thereof. It is indeed possible to employ an amount outside the range mentioned, for example 0.5 organyl equivalent per mole of rhenium compound, but this offers no particular advantages.

The reaction of the oxorhenium compound with the organylating agent as a rule takes place in an aprotic solvent. Ethers, such as dialkyl ethers and cyclic ethers, ether mixtures and mixtures of ethers with aliphatic and/or aromatic hydrocarbons, are suitable. Since Grignard reagents are conventionally prepared in an ethereal solvent, it is particularly expedient also to carry out the organylation of the rhenium oxo compound in the same solvent system. However, it may also be advantageous to replace an ethereal solvent present during the preparation of the Grignard reagent with a non-ethereal aprotic solvent before or after the reaction with the oxorhenium compound.

If the catalyst composition comprises no excess of organylating agent or a small excess does not noticeably interfere with the subsequent oxidation reaction, such a catalyst composition, especially if this is free from ethereal solvents, can be employed directly in the oxidation reaction.

While the composition K1 in general is a solvent-containing system, the catalyst system K3 is a catalyst system which has been at least partly freed from solvents and/or at least partly freed from inorganic constituents. The solvents are separated off in a manner known per se, conventionally by distilling off under reduced pressure. Inorganic constituents present in a solvent-containing catalyst system K1 can also be separated off by means of conventional methods for solid-liquid separation, including filtration methods, or by dissolving the inorganic constituents which are insoluble in an organic solvent system contained in K1 in an aqueous system, which is added, with subsequent phase separation and if required extraction with a water-insoluble or sparingly water-soluble organic solvent.

If the organylating agent is employed in excess for organylation of the rhenium compound, it is advisable to destroy this excess after the reaction by addition of a protic agent, and if required also to separate off the solids which thereby precipitate out from the organylrhenium oxide formed. The protic agent is expediently employed in at least the stoichiometric amount. Examples of suitable protic agents are solvents from the series consisting of water, alcohols, carboxylic acids, silicas, boric acids and mineral acids. The catalyst composition K2 is formed by decomposition of the excess organylating agent, which can take place before or after solvents and/or insoluble constituents are separated off. Instead of a liquid agent for decomposition of excess organylating agent, it is also possible to use solids which contain either hydroxyl functions, examples are silicas and aluminum oxides, and/or water adsorbed on their surface. One advantage of such solid decomposition agents is that these can easily be separated off from the catalyst composition K2, a catalyst composition K4 being formed. If required, all or some of the solvent contained in the composition K4 can also have been removed therefrom or replaced by another solvent. The removal of the solvent is necessary in particular if the oxidation reaction is to be carried out in a different solvent system to that from the preparation of the catalyst composition contained therein.

The preparation of the catalyst composition comprising at least one organylrhenium compound can be carried out in the presence of a Lewis-base compound which serves as a ligand for the organylrhenium oxide; in this case, d in the general formula RₐRe_{b}O_{c}L_{d} is not 0. Suitable ligands are aliphatic and aromatic amines, and in particular N-heteroaromatic compounds. Those ligands which are not themselves attacked under the conditions of the subsequent oxidation reaction or other reactions in the presence of a catalyst of the formula RₐRe_{b}O_{c}L_{d} and thus reduce the activity of the catalyst or lead to undesirable by-products are expediently employed.

The process according to the invention using a catalyst composition according to the invention can be used for the oxidation of very different organic substrates. Such oxidation reactions have already been described in the prior art, but the organyloxorhenium compound used as a catalyst, such as methyltrioxorhenium (MTO), was employed in the pure form. Examples of oxidizable substrates are olefins, which can be epoxidized and/or hydroxylated to vicinal diols, depending on the temperature and solvent conditions used. The principle and the reaction conditions are to be found, by way of example, in EP-B 0 380 085. In the process of this document, complexes of organylrhenium oxides with Lewis-base ligands L in purified form were also employed as a catalyst for the epoxidation or hydroxylation of olefins.

In the process according to the invention, in contrast, it is possible to use a catalyst composition according to K1, K2, K3 or K4, wherein this can already comprise a Lewis-base compound in complexed form or such a complex is first formed in situ by the addition of a ligand L during the use of the catalyst composition, as a catalyst for the oxidation reactions according to the invention without isolation of a catalytically active organyloxorhenium oxide or complex thereof contained therein. According to a preferred embodiment, both a catalyst composition to be used according to the invention and a Lewis-base compound are added to the reaction system for carrying out the oxidation, the catalytically active complex being formed in situ.

The Lewis-base compounds are preferably nitrogen bases, in particular nitrogen-containing heterocyclic compounds, such as, in particular, pyridine, pyrazole or dipyridine, it also being possible for these heterocyclic compounds to be substituted. Further suitable nitrogen bases are 1-azabicyclo[2,2,2]octane and various aromatic amines, the suitability of the latter depending in part on what reaction conditions are desired, since these aromatic amines can themselves be oxidized, depending on the reaction conditions chosen.

According to a specific embodiment of the invention, secondary amines are oxidized to disubstituted hydroxylamines. Aliphatic secondary amines, wherein the alkyl radicals bonded to the nitrogen can also contain one or more substituents, can be employed. According to a particularly preferred embodiment, the amine to be hydroxylated is a bis(hydroxyalkyl)amine, for example bis(2-methoxyethyl)aamine, bis(2-ethoxyethyl)amine, bis(2-n- or iso-propoxyethyl)amine and bis(2-n-butoxyethyl)amine and the analogous bis(alkoxypropyl)- and bis(alkoxybutyl)amines. Secondary amines with an aliphatic and an aromatic radical, such as N-methoxyethylaniline, can also be converted suitably into the corresponding hydroxylamines.

A further class of oxidation reactions which are accessible according to the invention is based on the Baeyer-Villiger reaction, wherein cyclic ketones, such as cyclopentanone or cyclohexanone, which can be substituted in their turn, are oxidized to the corresponding lactones.

Aromatics, in particular polynuclear aromatics, such as naphthalene, alkylnaphthalenes, anthracenes and alkylanthracenes, can also be converted into the corresponding quinone substances using a catalyst composition according to the invention.

Further substrates which can be oxidized with a peroxo compound, such as, in particular, hydrogen peroxide, in the presence of a catalyst composition according to the invention are: alkines, from which carboxylic acids or α-diketones are formed; organic sulfides, which can be converted into the sulfoxides or sulfones; and the oxidation of aromatic amines to nitrosoamines, and of aromatic N,N-dialkylamines to the corresponding N-oxides.

Apart from as a catalyst for oxidation reactions, the catalyst compositions according to the invention, like the known organylrhenium oxides isolated, are also suitable as a catalyst for aldehyde olefination, olefin metathesis and for Diels-Alder reactions.

The oxidation reactions according to the invention are carried out in a solvent system in a manner known per se, and the solvent system can be single- or multiphase and can also comprise water. However, the water content is expediently kept low in order to avoid or keep low a hydrolytic decomposition of the catalytically active system.

The oxidizing agent in the process according to the invention is a peroxo compound, in particular a source for hydrogen peroxide which is capable of releasing hydrogen peroxide under the reaction conditions, or hydrogen peroxide directly. Hydrogen peroxide is preferably employed as an aqueous solution, in particular as an aqueous solution with an H₂O₂ content of 35 to 80 wt. %. Among the sources for hydrogen peroxide there may be mentioned per-salts, such as sodium perborate monohydrate, sodium perborate tetrahydrate, sodium percarbonate, persulfates and persilicates. If basic per-salts are used, the pH must be lowered, where appropriate, before contact with the catalyst composition. H₂O₂ adducts, for example those on urea (=percarbamide) or on silicas, can also be employed. The oxidizing agent is conventionally employed in at least the stoichiometrically required amount, but a certain excess has proved advantageous in many cases.

The invention also provides a catalyst composition, in particular suitable for carrying out oxidation reactions, comprising at least one organylrhenium oxide of the formula RₐRe_{b}O_{c}L_{d}, wherein R represents an organyl radical, a represents an integer from 1 to 6, b represents an integer from 1 to 4, c represents an integer from 1 to 12, d represents an integer from 0 to 4, L represents a Lewis-base ligand and Re is penta- to heptavalent, and present as the catalyst composition K1 or a catalyst composition K2 to K4 obtained therefrom, characterized in that it has been obtained by a process comprising organylation of an oxorhenium compound of the general formula ReOₘAₙ, wherein Re is penta- to heptavalent, A represents an anionic leaving group, m represents an integer 1, 2 or 3 and n represents an integer 0, 1, 2 or 3, with a Grignard compound of the formula RMgHal or RZnHal, wherein R represents an organyl radical, in particular alkyl, and Hal represents a halogen, in particular chlorine, and wherein 0.5 to 10 mol of the Grignard compound are employed per mol of ReOₘAₙ, in an aprotic solvent system in the presence or absence of a Lewis-base ligand compound L, a catalyst composition K1 being obtained, decomposition of excess Grignard compound in the catalyst composition K1 by addition of a protic agent, a catalyst composition K2 being obtained, at least partial removal of solvents and/or inorganic constituents from K1 or K2, a catalyst composition K3 or K4 respectively being obtained, wherein the preparation of K1 to K4 excludes a sublimation, crystallization or distillation of the organylrhenium oxides of the formula RₐRe_{b}O_{c}L_{d} contained therein.

Preferred embodiments of the catalyst composition, reference being made to the above statements in respect of the reaction partners for the preparation of the catalyst composition K1 to K4.

The catalyst composition according to the invention in the embodiment K1 to K4 prepared using a Grignard compound as a rule comprises several organyloxorhenium compounds. It is an advantage of the catalyst composition according to the invention that the organylrhenium oxides contained therein do not have to be isolated out of the substance mixture and purified in order to be able to be used effectively as a catalyst. A further advantage lies in the easy accessibility and problem-free handling of the Grignard reagents.

### DETAILED EMBODIMENT OF THE INVENTION

The invention is illustrated further with the aid of the following examples.

### EXAMPLES 1.1 to 1.12

Preparation of a Catalyst Composition and Use Thereof for Epoxidation of Cyclooctene.

A solution of Re₂O₇ in tetrahydrofuran was employed as the oxorhenium compound (c=0.04 mmol Re₂O₇/ml) (=solution A). Methylmagnesium chloride in tetrahydrofuran (c=3.0 mol/l) (=solution B) was employed as the organylating agent.

Preparation of the Catalyst Composition

In each case 0.5 ml (R1-R4) or 2.5 ml (R5-R12) of solution A were metered into glass reactors which had been rendered inert. In each case 16 μl pyridine were added to reactors R4 to R6 and in each case 81 μl to reactors R10 to R12. After cooling to 0° C., solution B was added. The molar ratio of methylmagnesium chloride to rhenium heptoxide follows from table 1. After a reaction time of 2 h, the mixture was heated to 25° C. 4 ml 10 wt. % aqueous NH₄Cl solution were added to each reactor for the purpose of destroying excess/unreacted methylmagnesium chloride. After 5 min each batch was extracted with 4 ml CH₂Cl₂. The organic phase separated off is a catalyst composition K4 freed from inorganic constituents. If present, the pyridine added functioned at least partly as the ligand L.

The particular organic phase was transferred into 12 new reactors R1 to R12, and the solvents CH₂Cl₂ and tetrahydrofuran were then removed in vacuo at 25° C. Catalyst compositions K4 substantially freed from inorganic constituents and solvents were obtained.

Epoxidation

The residues in R1 to R12 were in each case taken up in 4 ml CH₂Cl₂, and in each case 1.8 ml H₂O₂ solution (35 wt. %) and 80 μl pyridine and 160 μl tetradecane (=internal standard for the GC evaluation) were added. In each case 1.0 ml cyclooctene was metered in with moderate cooling (approx. 19° C.). After a reaction time of 20 h samples were taken, 1 ml methylene chloride and a trace of MnO₂ were added and, after decomposition of the remaining H₂O₂, the organic phase was filtered over silica gel and analysed by means of GC (column: CP-SIL 13CB WCOT Fused Silica).

Table 1 shows the molar amounts employed in the preparation of the catalyst compositions, the amount of catalyst in the epoxidation and the yields of cyclooctene oxide obtained in the epoxidation:

TABLE 1

| | Methylation | | | Yield of |
|---|---|---|---|---|
| Reactor | Re₂O₇¹⁾ | Pyridine | CH₃MX | cyclooctene oxide |
| no. R | mol %_{Re}²⁾ | mol/mol Re₂O₇ | mol/mol Re₂O₇ | (%) |
| 1 | 0.5 | | 1 | 65.4 |
| 2 | 0.5 | | 3 | 92.4 |
| 3 | 0.5 | | 5 | 96.0 |
| 4 | 0.5 | 10 | 1 | 72.7 |
| 5 | 0.5 | 10 | 3 | 90.4 |
| 6 | 0.5 | 10 | 5 | 94.7 |
| 7 | 2.6 | | 1 | 95.0 |
| 8 | 2.6 | | 3 | 96.2 |
| 9 | 2.6 | | 5 | 95.3 |
| 10 | 2.6 | 10 | 1 | 97.1 |
| 11 | 2.6 | 10 | 3 | 97.0 |
| 12 | 2.6 | 10 | 5 | 95.9 |

¹⁾Re₂O₇ was employed in the methylation
²⁾The mol % Re stated is the amount of Re employed, based on the olefin to be epoxidized

### EXAMPLES 2.1 to 2.12

Examples 2.1 to 2.12 were carried out analogously to examples 1.1 to 1.12, but a methyllithium-lithium bromide complex in diethyl ether (c =1.5 mol/l) was employed as the organylating agent. Table 2 shows the molar amounts employed in the preparation of the catalyst compositions and the yields of cyclooctene oxide obtained in the epoxidation:

TABLE 2

| | Methylation | | | Yield of |
|---|---|---|---|---|
| Reactor | Re₂O₇¹⁾ | Pyridine | LiCH₃ | cyclooctene oxide |
| no. | mol-%_{Re}²⁾ | mol/mol Re₂O₇ | mol/mol Re₂O₇ | (%) |
| 1 | 0.5 | | 1 | 96.3 |
| 2 | 0.5 | | 3 | *) |
| 3 | 0.5 | | 5 | *) |
| 4 | 0.5 | 10 | 1 | 76.9 |
| 5 | 0.5 | 10 | 3 | *) |
| 6 | 0.5 | 10 | 5 | 82.8 |
| 7 | 2.6 | | 1 | 94.1 |
| 8 | 2.6 | | 3 | 92.6 |
| 9 | 2.6 | | 5 | 95.3 |
| 10 | 2.6 | 10 | 1 | 92.3 |
| 11 | 2.6 | 10 | 3 | 96. |
| 12 | 2.6 | 10 | 5 | 94.3 |

¹⁾Re₂O₇ was employed in the methylation
²⁾The mol % Re stated is the amount of Re employed, based on the olefin to be epoxidized
*) Experimental error

### COMPARISON EXAMPLES CE1 to CE6

The epoxidation of cyclooctene was carried out in a manner analogous to that in the examples according to the invention, but instead of a catalyst composition according to the invention the following substances or reaction mixtures were employed.
CE1: MTO (=pure methyltrioxorhenium oxide)
CE2: equimolar mixture of MTO and KReO₄
CE3: Re₂O₇
CE4: KReO₄
CE5 and CE6: Reaction mixture obtained by bringing KReO₄ into contact with 1 or 5 equivalents respectively of CH₃MgCl per equivalent of Re, treatment with aqueous NH₄Cl solution, extraction with CH₂Cl₂, phase separation and use of the organic phase, comprising “KReO₄ methylated”.

Table 3 shows the results

TABLE 3

| | | | | Yield of |
|---|---|---|---|---|
| CE | | Mol % Re based | CH₃MgCl | cyclooctene |
| no. | Re catalyst | on olefin | mol/mol Re | oxide (%) |
| 1 | MTO | 0.5 | — | 94.8 |
| 2 | MTO/KReO₄ | 0.5 | — | 89.1 |
| | (1:1) | | | |
| 3 | Re₂O₇ | 0.5 | — | 0.2 |
| 4 | KReO₄ | 0.5 | — | 0.2 |
| 5 | “KReO₄ | 2.5 | 1 | 0.2 |
| | methylated” | | | |
| 6 | “KReO₄ | 2.5 | 5 | 0 |
| | methylated” | | | |

On comparison of comparison example CE1 with examples 1.1 to 1.3 according to the invention and alternatively 2.1, it can be seen that the catalytic activity is substantially the same. By lowering the amounts of MTO employed by replacement of half by the Re-equivalent amount of KReO₄ while maintaining the molar amount of Re compounds employed in the oxidation, the epoxidation yield decreases somewhat (CE2). This drop in yield was to be expected in view of the result of comparison example CE4, since KReO₄ proved to be catalytically inactive. As follows from CE5 and CE6, KReO₄ evidently cannot be alkylated under the alkylation conditions according to the invention, since a reaction mixture obtained in this way is catalytically inactive.

The comparison of the results of examples 1.2 and 1.3 or 2.1 according to the invention with comparison example CE2 is completely surprising, because the catalytic activity of the catalyst compositions according to the invention under the conditions of the examples proved to be even higher than that of the mixture of pure MTO and KReO₄ (molar ratio 1:1). According to the prior art (see pages 3210-3211 of the review article cited), MTO and perrhenate are theoretically formed in an equivalent amount on methylation of Re₂O₇ with an alkylating agent; in reality, the content of MTO is significantly lower due to secondary reactions, such as reduction reactions, depending on the conditions chosen; furthermore, losses of MTO cannot be avoided during the isolation of the MTO from the reaction mixture, which is considered necessary. In contrast, in the catalyst composition according to the invention other alkylrhenium oxides produced during the alkylation of Re₂O₇ evidently also display a catalytic action and furthermore losses of MTO and/or other catalytically active alkylrhenium oxides are avoided due to the direct use of the catalyst composition.

### EXAMPLES 3.1 to 3.6

Epoxidation of 1-Hexene

Preparation of the catalyst compositions for examples 3.1 to 3.6 and 4.1 to 4.6

The following solutions of Re₂O₇ in tetrahydrofuran (THF) were employed:
Variant V1: 813.8 mg Re₂O₇ in 42 ml THF (=0.040 mol/l)
Variant V2: 542.5 mg Re₂O₇ in 42 ml THF (=0.0267 mol/l)
Variant V3: 406.9 mg Re₂O₇ in 42 ml THF (=0.020 mol/l)
Variant V4: 203.3 mg Re₂O₇ in 42 ml THF (=0.010 mol/l)

The catalyst solution is prepared with exclusion of oxygen and moisture. 2.5 ml of the solution V1, V2, V3 or V4 are initially introduced into a 25 ml two-necked flask with a magnetic stirrer and internal thermometer and cooled to 0° C. A 3 molar solution of methylmagnesium chloride in tetrahydrofuran is cautiously added to this, and in particular per mol of Re₂O₇ 3 mol (A), 4.5 mol (B), 6 mol (C) or 12 mol (D) of CH₃MgCl.

An immediate change in colour of the solution to brown takes place. The solution is subsequently stirred in the dark at 0C for a total of 2 h and, after removal of the ice bath, is stirred for a further 5 min and then allowed to warm to room temperature. For the hydrolysis, 4 ml of a 10% ammonium chloride solution are added and the mixture is stirred for 5 min. Extraction with 4 ml methylene chloride is then carried out. The lower, organic phase which has been separated off, 6.5 ml in total, is employed as the catalyst solution for the oxidation. The amount (ml) of these catalyst solutions employed is shown in tables 4 and 5.

Oxidation of 1-Hexene

1 ml (7.64 mmol) 1-hexene and 80 μl (12.97 mol %) pyridine were initially introduced into a 25 ml three-necked flask at room temperature and, unless stated otherwise, 6.5 ml of the catalyst solution stated in table 4 were added. 0.9 ml (18.56 mmol, that is to say 2.43 mol H₂O₂ per mol of 1-hexene) of aqueous hydrogen peroxide (70 wt. %) was slowly added to the solution, while stirring, such that the temperature did not rise above 30° C. The GC crude yield of hexene oxide after a reaction time of 26 h follows from the last column of table 4.

### RE EXAMPLE 3.5:

0.9 ml (18.56 mmol) of aqueous hydrogen peroxide (70 wt. %) were slowly added to 6.5 ml of catalyst solution V1A at room temperature, while stirring, an emulsion forming. 1 ml (7.64 mmol) 1-hexene and 80 μl (12.97 mol %) pyridine were initially introduced into a 25 ml three-necked flask at room temperature and the emulsion comprising H₂O₂ and catalyst was slowly added such that the internal temperature did not rise above 30° C. The GC crude yield after a reaction time of 26 h is 93.2% of hexene oxide.

### RE EXAMPLE 3.6

The catalyst composition formed from VIC and freed from the solvent was employed. The reaction was thus carried out in the absence of a solvent.

### RE EXAMPLE 3.7

The oxidation was carried out at a molar ratio of H₂O₂/olefin of 1.25, that is to say a considerably lower ratio than in examples 3.1 to 3.6. As the table shows, however, a considerably higher yield is achieved than in example 3. 1, with the same type and amount of catalyst but a higher molar ratio of H₂O₂/olefin.

TABLE 4

| | Catalyst | | | CH₃MgCl | | Hexene |
|---|---|---|---|---|---|---|
| Ex. | solution | | Re₂O₇ | eq. to | | oxide |
| no. | in ml | Variant | mol %^{#} | Re₂O₇ | CH₂Cl₂^{##} ml | (GC %) |
| 3.1 | 6.5 | V1A | 1.30 | 3 | — | 52.5 |
| 3.2 | 6.5 | V1A | 1.30 | 3 | 4 | 82.2 |
| 3.3 | 6.5 | V1A | 0.651 | 6 | — | 78.2 |
| 3.4 | 6.5 | V1C | 1.30 | 6 | 4 | 87.2 |
| 3.5 | 6.5* | V1A | 1.30 | 6 | — | 93.2 |
| 3.6 | 6.5** | V1C | 1.30 | 6 | — | 82.6 |
| 3.7 | 6.5*** | V1A | 1.30 | 3 | — | 78.8 |

*), **) and ***) see notes before the table
#mol % Re₂O₇ based on 1-hexene
##additional addition before the reaction

### EXAMPLES 4.1 to 4.6

### Oxidation of N,N-bis-(2-methoxyethyl)-amine

5 ml (3.71 g; 34.16 mmol) N,N-bis-(2-methoxyethyl)-amine, 15 ml CH₂Cl₂ and 32 μl (1.16 mol %) pyridine were initially introduced into a 25 ml three-necked flask and the amount and nature of the catalyst solution stated in table 5 was added. The solution was now cooled to −15° C. and a total of 1.91 ml (1.3 eq.; 44.41 mol) of aqueous hydrogen peroxide (70 wt. %) were slowly added, while stirring, such that the internal temperature did not rise above 5° C. The reaction is very exothermic, especially in the initial phase. When the addition had ended, the mixture was subsequently stirred at approx. −15° C. for a further 4 h. The solution is then two-phase. The table shows the yields, determined by GC evaluation, of bis-N,N-(2-methoxyethyl)-hydroxylamine (DMEHA), unreacted educt and nitrone formed by superoxidation.

TABLE 5

| | Catalyst solution | | Re₂O₇ | CH₃MgCl | CH₂Cl₂ | Educt | DMEHA | Nitrone |
|---|---|---|---|---|---|---|---|---|
| Ex. no. | V in ml | Variant | (mol %) | eq. to Re₂O₇ | (ml) | (GC %) | (GC %) | (GC %) |
| 4.1 | 2.22 | V1A | 0.10 | 3 | 15 | 6.39 | 82.5 | 11.1 |
| 4.2 | 2.22 | V1C | 0.10 | 6 | 15 | 0.4 | 83.3 | 16.0 |
| 4.3 | 2.22 | V2B | 0.066 | 4.5 | 15 | 1.8 | 83.6 | 13.8 |
| 4.4 | 1.11 | V1C | 0.05 | 6 | 15 | 4.39 | 81.86 | 13.75 |
| 4.5 | 2.22 | V3C | 0.05 | 6 | 15 | 1.2 | 82.2 | 11.5 |
| 4.6 | 2.22 | V4C | 0.025 | 6 | 15 | 6.4 | 79.5 | 12.9 |

*GC % after a subsequent stirring time of 4 h at −15° C.

### COMPARISON EXAMPLE CE7

Amine oxidation was catalysed with 0.05 mol % MTO. 82% DMEHA, 1.5% educt and 14% nitrone were obtained.

Further variations and modifications of the foregoing will be apparent to those skilled in the art and are intended to be encompassed by the Claims appended hereto.

German priority application (103 42 150.5) is relied on and incorporated herein by reference.

1. A process for the oxidation of an organic substrate comprising reacting the organic substrate, in a solvent, with a peroxo compound, in the presence of an organylrhenium oxide of the formula RₐRe_{b}O_{c}L_{d},
wherein R is an organyl radical, a is an integer from 1 to 6, b is an integer from 1 to 4, c is an integer from 1 to 12, d is an integer from 0 to 4, L is a Lewis-base ligand and Re is penta- to heptavalent, as a catalyst,
wherein the catalyst is selected from the group consisting of K1, K2, K3 and K4,
wherein composition K1 is prepared by reacting an oxorhenium compound of the formula ReOₘAₙ, wherein Re is penta- to heptavalent, A represents an anionic leaving group, m represents the number 1, 2 or 3 and n represents the number 0, 1, 2 or 3, with 1 to 10 organyl equivalents per mole of ReOₘAₙ of an organylating agent of an element selected from the group consisting of Li, Na, K, Mg, Al and Zn in an aprotic solvent in the presence or absence of a Lewis-basic ligand,
wherein K2 is prepared by the addition of a protic agent to K1 comprising excess organylating agent,
and K3 and K4 are prepared by at least partial removal of the solvents and/or inorganic constituents from K1 and K2 respectively, but the preparation of K1 to K4 excludes a sublimation, crystallization or distillation of the organylrhenium oxides of the formula RₐRe_{b}O_{c}L_{d} contained therein.

2. The process according to claim 1, where the peroxo compound is hydrogen peroxide.

3. The process according to claim 1, wherein the substrate is an olefin that is epoxidized or hydroxylated to a diol.

4. The process according to claim 1, wherein the substrate is an amine that is oxidized.

5. The process according to claim 4, wherein the amine is a secondary amine that is oxidized to a disubstituted hydroxylamine.

6. A process according to claim 1, wherein the substrate is a cyclic ketone that is oxidized to a lactone by the Baeyer-Villiger process.

7. The process according to claim 1, wherein the substrate is an aromatic.

8. The process according to claim 7, wherein the substrate is a polynuclear aromatic which is oxidized to a quinone.

9. The process according to claim 1, wherein the oxorhenium compound is a compound selected from the group consisting of ReO₃, Re₂O₇, Hal-ReO₃, Hal₂ReO₂ and ReO₃—O-acyl, wherein Hal represents F, Cl, Br or I and acyl represents a group selected from the group consisting of R—C(O)—, aryl-SO₂—, —P(O)(OR′)₂, —Si(OR′)₃, —P(O)R″₂ and —SiR″₃, wherein R represents optionally fluorinated alkyl and R′ and R″ represent alkyl, cycloalkyl, arylalkyl or aryl, which can also be fluorinated.

10. The process according to claim 9, wherein the substrate is an olefin, amine, cyclic ketone or aromatic.

11. The process according to claim 1, wherein the organylating agent is a compound selected from the group consisting of LiR, NaR, KR, MgR₂, RMgHal, ZnR₂, AlR₃ and Al(O)R, wherein R represents linear, branched or cyclic alkyl.

12. The process according to claim 1 1, wherein R is unbranched C₁-C₄ alkyl, benzyl or aryl, which can be alkyl substituted.

13. The process according to claim 1, wherein the organylating agent is a compound selected from the group consisting of lithium-alkyl compounds and Grignard reagents.

14. The process according to claim 13, wherein said compound is an alkylmagnesium halide.

15. The process according to claim 14, wherein said compound is methyl-magnesium chloride.

16. The process according to claim 1, wherein 1 to 5 organyl equivalents are employed per mol of oxorhenium compound.

17. The process according to claim 1, wherein >1 to 3 organyl equivalents are employed per mol of oxorhenium compound.

18. The process according to claim 1, wherein the organylation of the oxorhenium compound and/or the oxidation reaction is carried out in the presence of a nitrogen base as the ligand L which can form complexes of the formula RₐRe_{b}O_{c}L_{d} with the organylrhenium oxides contained in the catalyst.

19. The process according to claim 1, further comprising decomposing excess organylating agent contained in the reaction mixture K1 by adding an at least stoichiometric amount of a solvent selected from the group consisting of water, alcohols, carboxylic acids, silicas, boric acids and mineral acids, and forming the catalyst composition K2.

20. The process according to claim 1, further comprising decomposing excess organylating agent in K1 composition by adding an active amount of a particulate solid with hydroxyl functions or adsorbed water, separating off the solid from catalyst composition K2 obtained thereby, which has been diluted with a solvent if required, and thereby obtaining catalyst composition K4.

21. The process according to claim 1, further comprising at least partly freeing a catalyst composition K3 or K4 from solvent and inorganic constituents, and converting K1 into K4 by an addition of water and an extraction of the aqueous solution or suspension with an organic solvent.

22. A catalyst composition suitable for carrying out oxidation reactions comprising at least one organylrhenium oxide of the formula RₐRe_{b}O_{c}L_{d},
wherein R represents an organyl radical, a represents an integer from 1 to 6, b represents an integer from 1 to 4, c represents an integer from 1 to 12, d represents an integer from 0 to 4 and L represents a Lewis-base ligand and Re is penta- to heptavalent, and present as the catalyst composition K1,
wherein said catalyst composition K1 is obtained by a process comprising organylation of an oxorhenium compound of the general formula ReOₘAₙ, wherein Re is penta- to heptavalent, A represents an anionic leaving group, m represents an integer 1, 2 or 3 and n represents an integer 0, 1, 2 or 3, with a Grignard compound of the formula RMgHal or RZnHal, wherein R represents an organyl radical, and Hal represents a halogen, and wherein 0.5 to 10 mol of the Grignard compound are employed per mol of ReOₘAₙ, in an aprotic solvent system in the presence or absence of a Lewis-base ligand compound L, wherein the preparation of K1 excludes a sublimation, crystallization or distillation of the organylrhenium oxides of the formula RₐRe_{b}O_{c}L_{d} contained therein.

23. The catalyst composition according to claim 22,wherein the oxorhenium compound is selected from the group consisting of ReO₃, Re₂O₇, Hal-ReO₃, Hal₂ReO₂ and ReO₃—O-acyl, wherein acyl represents an optionally substituted alkanoyl group, aryl-SO₂—, —P(O)(OR′)₂ , —Si(OR′)₃, —P(O)R″₂ and —SiR″₃ wherein R′ and R″ represent alkyl, cycloalkyl, arylalkyl or aryl, which can also be fluorinated.

24. The catalyst composition according to claim 23, further comprising at least one nitrogen base L which is capable of complexing the organylrhenium oxides contained in the catalyst composition.

25. A catalyst composition suitable for carrying out oxidation reactions comprising at least one organylrhenium oxide of the formula RₐRe_{b}O_{c}L_{d},
wherein R represents an organyl radical, a represents an integer from 1 to 6, b represents an integer from 1 to 4, c represents an integer from 1 to 12, d represents an integer from 0 to 4 and L represents a Lewis-base ligand and Re is penta- to heptavalent, and present as the catalyst composition K2 obtained by a process comprising organylation of an oxorhenium compound of the general formula ReOₘAₙ, wherein Re is penta- to heptavalent, A represents an anionic leaving group, m represents an integer 1, 2 or 3 and n represents an integer 0, 1, 2 or 3, with a Grignard compound of the formula RMgHal or RZnHal, wherein R represents an organyl radical, and Hal represents a halogen, and wherein 0.5 to 10 mol of the Grignard compound are employed per mol of ReOₘAₙ, in an aprotic solvent system in the presence or absence of a Lewis-base ligand compound L,
to thereby obtain catalyst composition K1,
decomposing excess Grignard compound in catalyst composition K1 by addition of a protic agent to thereby obtain catalyst composition K2, wherein the preparation of K1 to K2 excludes a sublimation, crystallization or distillation of the organylrhenium oxides of the formula RₐRe_{b}O_{c}L_{d} contained therein.

26. The catalyst composition according to claim 25, wherein the oxorhenium compound is a compound selected from the group consisting of ReO₃, Re₂O₇ Hal-Re0₃, Hal₂ReO₂ and ReO₃—O-acyl, wherein acyl represents an optionally substituted alkanoyl group, aryl-SO₂—, —P(O)(OR′)₂ , —Si(OR′)₃, —P(O)R″₂ and —SiR″₃, wherein R′ and R″ represent alkyl, cycloalkyl, arylalkyl or aryl, which can also be fluorinated.

27. The catalyst composition according to claim 25, wherein an organylmagnesium chloride, wherein organyl is chosen from the series consisting of methyl, ethyl, n-propyl, isopropyl, n-butyl, sec-butyl and allyl is the Grignard compound.

28. The catalyst composition according to claim 25, wherein 1 to 5 mol of Grignard compound is employed per mol of oxorhenium compound.

29. The catalyst composition according to claim 25, wherein it additionally comprises at least one nitrogen base L which is capable of complexing the organylrhenium oxides contained in the catalyst composition.

30. A catalyst composition suitable for carrying out oxidation reactions comprising at least one organylrhenium oxide of the formula RₐRe_{b}O_{c}L_{d},
wherein R represents an organyl radical, a represents an integer from 1 to 6, b represents an integer from 1 to 4, c represents an integer from 1 to 12, d represents an integer from 0 to 4 and L represents a Lewis-base ligand and Re is penta- to heptavalent, and present as the catalyst composition K3 or K4,
wherein said catalyst is obtained by a process comprising organylation of an oxorhenium compound of the general formula ReOₘAₙ, wherein Re is penta- to heptavalent, A represents an anionic leaving group, m represents an integer 1, 2 or 3 and n represents an integer 0, 1, 2 or 3, with a Grignard compound of the formula RMgHal or RZnHal, wherein R represents an organyl radical, and Hal represents a halogen, and wherein 0.5 to 10 mol of the Grignard compound are employed per mol of ReOₘAₙ, in an aprotic solvent system in the presence or absence of a Lewis-base ligand compound L,
to thereby obtain a catalyst composition K1, decomposing excess Grignard compound in the catalyst composition K1 by addition of a protic agent, a catalyst composition K2 being obtained thereby, at least partially removing solvents and/or inorganic constituents from K1 or K2, to thereby obtain a catalyst composition K3 or K4 respectively, wherein the preparation of K1 to K4 excludes a sublimation, crystallization or distillation of the organylrhenium oxides of the formula RₐRe_{b}O_{c}L_{d} contained therein.

31. A catalyst composition according to claim 30, wherein the oxorhenium compound is a compound selected from the group consisting of ReO₃, Re₂O₇, Hal-ReO₃, Hal₂ReO₂ and ReO₃—O-acyl, wherein acyl represents an optionally substituted alkanoyl group, aryl-SO₂—, —P(O)(OR′)₂ , —Si(OR′)₃, —P(O)R″₂ and —SiR″₃ wherein R′ and R″ represent alkyl, cycloalkyl, arylalkyl or aryl, which can also be fluorinated.

32. A catalyst composition according to claim 30, where the Grignard compound is an organylmagnesium chloride, wherein organyl is chosen from the series consisting of methyl, ethyl, n-propyl, isopropyl, n-butyl, sec-butyl and allyl.

33. The catalyst composition according to claim 30, wherein 1 to 5 mol of Grignard compound were employed per mol of oxorhenium compound.

34. The catalyst composition according to claim 30, wherein it additionally comprises at least one nitrogen base L which is capable of complexing the organylrhenium oxides contained in the catalyst composition.

35. The catalyst composition according to claim 30 which is a catalyst composition K3 or K4 which has been freed from solvents and inorganic constituents.
